(19) United States
(12) Patent Application Publication
(10) Pub. No.: US 2026/0335625 A1
(43) Pub. Date: Oct. 29, 2026

(54) PERCUTANEOUS CIRCULATORY SUPPORT DEVICE WITH IMPROVED PUSHABILITY

(71) Applicant: BOSTON SCIENTIFIC SCIMED, INC., MAPLE GROVE, MN (US)

(72) Inventors: Chris Cullen, Galway (IE); Tim O'Connor, Galway (IE); Gavin O'Brien, Sutton (IE); Neil O'Connor, Galway (IE); Daniel J. Presteng, Corcoran, MN (US); Victor Kotov, St. Louis Park, MN (US)

(73) Assignee: BOSTON SCIENTIFIC SCIMED, INC., MAPLE GROVE, MN (US)

(21) Appl. No.: 19/656,031

(22) Filed: Apr. 23, 2026

Related U.S. Application Data
(60) Provisional application No. 63/793,907, filed on Apr. 24, 2025.

(51) Int. Cl. A61M 60/174; A61M 60/226; A61M 60/416; A61M 60/531

(52) U.S. Cl. CPC A61M 60/174; A61M 60/226; A61M 60/416; A61M 60/531

(57) ABSTRACT

A percutaneous circulatory support device includes a blood pump including a blood inlet and a blood outlet, and an elongate shaft extending proximally from the blood pump. An outer sheath surrounds the elongate shaft and has one or more longitudinal separation lines extending along a length of the outer sheath to permit splitting the outer sheath to remove the outer sheath from the elongate shaft.

### CROSS-REFERENCE TO RELATED APPLICATION

This application claims the benefit of U.S. patent application Ser. No. 63/793,907, filed Apr. 24, 2025, entitled “PERCUTANEOUS CIRCULATORY SUPPORT DEVICE WITH IMPROVED PUSHABILITY”, which is incorporated by reference herein in its entirety.

### TECHNICAL FIELD

The disclosure pertains to medical devices and more particularly to percutaneous circulatory support devices, such as left ventricular assist devices, having enhanced pushability, and methods for using such medical devices.

### BACKGROUND

A wide variety of medical devices have been developed for medical use including, for example, devices for delivering intravascular medical devices into a blood vessel of a patient. Percutaneous circulatory support devices including a blood pump can be advanced into a patient's vasculature along a guidewire to be positioned in the patient's heart. There is a tradeoff between providing the elongate shaft of the percutaneous circulatory support device with a sufficient outer diameter and/or rigidity to withstand high pushing forces during advancement through the vasculature without buckling, yet having a sufficiently small outer diameter to permit blood perfusion through the vasculature with the elongate shaft disposed therein. There is an ongoing need to provide alternative configurations of percutaneous circulatory support devices, and methods of advancing the percutaneous circulatory support devices into a patient's vasculature.

### SUMMARY

This disclosure provides design, material, manufacturing method, and use alternatives for medical devices. An example medical device includes a percutaneous circulatory support device comprising a blood pump, an elongate shaft extending proximally from the blood pump, and an outer sheath having a proximal end, a distal end, and one or more longitudinal separation lines extending along a length of the outer sheath. The blood pump may include a blood inlet and a blood outlet. In a first configuration, the elongate shaft is disposed within a lumen of the outer sheath, and in a second configuration, the outer sheath is configured to be split along the one or more longitudinal separation lines and withdrawn from over the elongate shaft.

Alternatively or additionally to the embodiment above, the elongate shaft has an outer diameter of 6 Fr or less.

Alternatively or additionally to the embodiments above, the outer sheath has an outer diameter of 11 Fr or more.

Alternatively or additionally to the embodiments above, the outer sheath includes first and second longitudinal separation lines configured to split the outer sheath into two segments.

Alternatively or additionally to the embodiments above, the one or more longitudinal separation lines are one or more score lines of reduced wall thickness.

Alternatively or additionally to the embodiments above, when in the first configuration, the outer sheath is not axially moveable relative to the elongate shaft.

Alternatively or additionally to the embodiments above, the distal end of the outer sheath has a flared shape configured to fit around a proximal end of the blood pump.

Alternatively or additionally to the embodiments above, the percutaneous circulatory support device further includes a grip disposed on the proximal end of the outer sheath, wherein proximal withdrawal of the grip initiates proximal retraction and dividing of the outer sheath into multiple pieces.

Alternatively or additionally to the embodiments above, the grip is connected to a ripcord configured to open the one or more longitudinal separation lines of the outer sheath.

Alternatively or additionally to the embodiments above, an inner diameter of the outer sheath is greater than the outer diameter of the elongate shaft.

Alternatively or additionally to the embodiments above, the one or more longitudinal separation lines define an area of weakness providing a preferential separation region.

Alternatively or additionally to the embodiments above, the blood pump includes a housing and an impeller rotatably positioned in the housing for conveying blood from a blood inlet to a blood outlet.

Alternatively or additionally to the embodiments above, the outer sheath has a length of 60 cm or more.

Alternatively or additionally to the embodiments above, the outer sheath has a distal end located proximate a distal end of the elongate shaft and the outer sheath has a proximal end located proximate a proximal end of the elongate shaft.

An example medical device includes a percutaneous circulatory support device comprising a blood pump, an elongate shaft extending proximally from the blood pump, and an outer sheath surrounding the elongate shaft. The outer sheath has a proximal end proximate a proximal end of the elongate shaft, a distal end proximate a distal end of the elongate shaft, and one or more longitudinal separation lines extending along a length of the outer sheath. The outer sheath is configured to be split along the one or more longitudinal separation lines and withdrawn from over the elongate shaft.

Alternatively or additionally to the embodiments above, the outer sheath is not fixedly secured relative to the elongate shaft.

Alternatively or additionally to the embodiments above, the distal end of the outer sheath is proximate a proximal end of the blood pump.

An example method of placing a percutaneous circulatory support device within a vasculature includes advancing a percutaneous circulatory support device through a vasculature of a patient, the percutaneous circulatory support device including a blood pump including a blood inlet and a blood outlet, an elongate shaft extending proximally from the blood pump, and an outer sheath surrounding the elongate shaft, the outer sheath having a proximal end proximate a proximal end of the elongate shaft, a distal end proximate a distal end of the elongate shaft, and one or more longitudinal separation lines extending along a length of the outer sheath, positioning the blood pump at a desired treatment location, thereafter, removing the outer sheath from the elongate shaft with the blood pump at the desired treatment location.

Alternatively or additionally to the embodiments above, the outer sheath further comprises at least one longitudinal separation line extending along a length of the outer sheath.

Alternatively or additionally to the embodiments above, further comprising dividing the outer sheath along the at least one longitudinal separation line to remove the outer sheath from the elongate shaft.

Alternatively or additionally to the embodiments above, the outer sheath is not fixedly secured relative to the elongate shaft.

The above summary of some embodiments, aspects, and/or examples is not intended to describe each embodiment or every implementation of the present disclosure. The figures and the detailed description which follows more particularly exemplify these embodiments.

### BRIEF DESCRIPTION OF THE DRAWINGS

The disclosure may be more completely understood in consideration of the following detailed description of various embodiments in connection with the accompanying drawings, in which:

FIG. 1 is a perspective view of an exemplary percutaneous circulatory support device including a percutaneous blood pump;

FIG. 2 shows a distal end region of the percutaneous circulatory support device of FIG. 1 including the blood pump;

FIG. 3 shows an enlarged view of an elongate shaft of the percutaneous circulatory support device of FIG. 1;

FIG. 4 illustrates an outer sheath being removed from the elongate shaft of FIG. 3; and

FIG. 5 illustrates a mechanism for removing the outer sheath of FIG. 4 according to one embodiment.

While aspects of the disclosure are amenable to various modifications and alternative forms, specifics thereof have been shown by way of example in the drawings and will be described in detail. It should be understood, however, that the intention is not to limit aspects of the disclosure to the particular embodiments described. On the contrary, the intention is to cover all modifications, equivalents, and alternatives falling within the spirit and scope of the disclosure.

### DETAILED DESCRIPTION

For the following defined terms, these definitions shall be applied, unless a different definition is given in the claims or elsewhere in this specification.

All numeric values are herein assumed to be modified by the term “about,” whether or not explicitly indicated. The term “about”, in the context of numeric values, generally refers to a range of numbers that one of skill in the art would consider equivalent to the recited value (e.g., having the same function or result). In many instances, the term “about” may include numbers that are rounded to the nearest significant figure. Other uses of the term “about” (e.g., in a context other than numeric values) may be assumed to have their ordinary and customary definition(s), as understood from and consistent with the context of the specification, unless otherwise specified.

Although some suitable dimensions, ranges, and/or values pertaining to various components, features and/or specifications are disclosed, one of skill in the art, incited by the present disclosure, would understand desired dimensions, ranges, and/or values may deviate from those expressly disclosed.

As used in this specification and the appended claims, the singular forms “a”, “an”, and “the” include plural referents unless the content clearly dictates otherwise. As used in this specification and the appended claims, the term “or” is generally employed in its sense including “and/or” unless the content clearly dictates otherwise. It is to be noted that in order to facilitate understanding, certain features of the disclosure may be described in the singular, even though those features may be plural or recurring within the disclosed embodiment(s). Each instance of the features may include and/or be encompassed by the singular disclosure(s), unless expressly stated to the contrary. For simplicity and clarity purposes, not all elements of the disclosure are necessarily shown in each figure or discussed in detail below. However, it will be understood that the following discussion may apply equally to any and/or all of the components for which there are more than one, unless explicitly stated to the contrary. Additionally, not all instances of some elements or features may be shown in each figure for clarity.

Relative terms such as “proximal”, “distal”, “advance”, “withdraw”, variants thereof, and the like, may be generally considered with respect to the positioning, direction, and/or operation of various elements relative to a user/operator/manipulator of the device, wherein “proximal” and “withdraw” indicate or refer to closer to or toward the user and “distal” and “advance” indicate or refer to farther from or away from the user. In some instances, the terms “proximal” and “distal” may be arbitrarily assigned in an effort to facilitate understanding of the disclosure, and such instances will be readily apparent to the skilled artisan. Other relative terms, such as “upstream”, “downstream”, “inflow”, and “outflow” refer to a direction of fluid flow within a lumen, such as a body lumen, a blood vessel, or within a device.

The term “extent” may be understood to mean a greatest measurement of a stated or identified dimension, unless the extent or dimension in question is preceded by or identified as a “minimum”, which may be understood to mean a smallest measurement of the stated or identified dimension. For example, “outer extent” may be understood to mean a maximum outer dimension, “radial extent” may be understood to mean a maximum radial dimension, “longitudinal extent” may be understood to mean a maximum longitudinal dimension, etc. Each instance of an “extent” may be different (e.g., axial, longitudinal, lateral, radial, circumferential, etc.) and will be apparent to the skilled person from the context of the individual usage. Generally, an “extent” may be considered a greatest possible dimension measured according to the intended usage, while a “minimum extent” may be considered a smallest possible dimension measured according to the intended usage. In some instances, an “extent” may generally be measured orthogonally within a plane and/or cross-section, but may be, as will be apparent from the particular context, measured differently—such as, but not limited to, angularly, radially, circumferentially (e.g., along an arc), etc. Additionally, the term “substantially” when used in reference to two dimensions being “substantially the same” shall generally refer to a difference of less than or equal to 5%.

The terms “monolithic” and “unitary” shall generally refer to an element or elements made from or consisting of a single structure or base unit/element. A monolithic and/or unitary element shall exclude structure and/or features made by assembling or otherwise joining multiple discrete elements together.

It is noted that references in the specification to “an embodiment”, “some embodiments”, “other embodiments”, etc., indicate that the embodiment(s) described may include a particular feature, structure, or characteristic, but every embodiment may not necessarily include the particular feature, structure, or characteristic. Moreover, such phrases are not necessarily referring to the same embodiment. Further, when a particular feature, structure, or characteristic is described in connection with an embodiment, it would be within the knowledge of one skilled in the art to affect the particular feature, structure, or characteristic in connection with other embodiments, whether or not explicitly described, unless clearly stated to the contrary. That is, the various individual elements described below, even if not explicitly shown in a particular combination, are nevertheless contemplated as being combinable or arrangeable with each other to form other additional embodiments or to complement and/or enrich the described embodiment(s), as would be understood by one of ordinary skill in the art.

For the purpose of clarity, certain identifying numerical nomenclature (e.g., first, second, third, fourth, etc.) may be used throughout the description and/or claims to name and/or differentiate between various described and/or claimed features. It is to be understood that the numerical nomenclature is not intended to be limiting and is exemplary only. In some embodiments, alterations of and deviations from previously-used numerical nomenclature may be made in the interest of brevity and clarity. That is, a feature identified as a “first” element may later be referred to as a “second” element, a “third” element, etc. or may be omitted entirely, and/or a different feature may be referred to as the “first” element. The meaning and/or designation in each instance will be apparent to the skilled practitioner.

The following description should be read with reference to the drawings, which are not necessarily to scale, wherein similar elements in different drawings are numbered the same. The detailed description and drawings are intended to illustrate but not limit the disclosure. Those skilled in the art will recognize that the various elements described and/or shown may be arranged in various combinations and configurations without departing from the scope of the disclosure. The detailed description and drawings illustrate example embodiments of the disclosure. However, in the interest of clarity and ease of understanding, while every feature and/or element may not be shown in each drawing, the feature(s) and/or element(s) may be understood to be present regardless, unless otherwise specified.

Patients with decreased cardiac output may be treated with a percutaneous circulatory support device, such as a left ventricular assist device (LVAD), which is positioned across the aortic valve into the left ventricle to increase the flow of oxygenated blood through the body. The placement of a percutaneous circulatory support device provides a challenge of advancing the device through the vasculature of the patient to the heart. Navigating tight and tortuous anatomy requires the application of a significant push force to ensure the blood pump, which is mounted at a distal end region of an elongate shaft of the percutaneous circulatory support device, progresses to the desired location smoothly. For successful navigation and placement, the elongate shaft must possess adequate pushability to avoid buckling or bending under the push force. However, this requirement for increased pushability, via a more robust elongate shaft, can impede blood flow after the percutaneous circulatory support device has been placed. Thus, a method and device for increasing pushability, while allowing for optimal blood flow post-placement is needed.

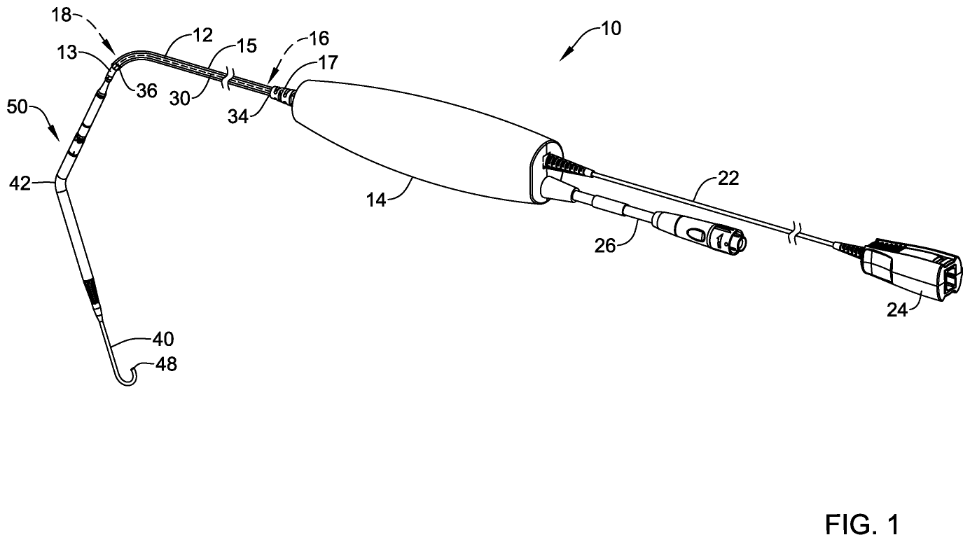

FIG. 1 illustrates a perspective view of a percutaneous circulatory support device 10 including a percutaneous blood pump 50 located at a distal end region thereof. The percutaneous circulatory support device 10 may be coupled to or include the blood pump 50, with an elongate shaft 12 of the percutaneous circulatory support device 10 extending proximally from the percutaneous blood pump 50 and a distal tip 40 extending distally from the blood pump 50. The distal tip 40 having and extending to a distal end 48. A distal portion of the blood pump 50 may be flexible, with a proximal portion being rigid. In some instances, the blood pump 50 may include a bend 42 along its length such that a first portion of the blood pump 50 extends along a first longitudinal axis and a second portion of the blood pump 50 extends along a second longitudinal axis, non-parallel to the first longitudinal axis. A proximal end 16 of the elongate shaft 12 may be coupled to a junction housing 14 and a distal end 18 of the elongate shaft 12 may be coupled to the percutaneous blood pump 50.

A removable outer sheath 15 is disposed over the elongate shaft 12, and includes an elongate tubular body extending from a proximal end 34 of the removable outer sheath 15 to a distal end 36 of the removable outer sheath 15. The outer sheath 15 may have at least one lumen defined therein that houses the elongate shaft 12 such that the outer sheath 15 circumferentially surrounds the elongate shaft 12. The outer sheath 15 is removable from the elongate shaft 12, for example, along one or more longitudinal separation lines 30. The outer sheath 15 is configured to add additional support and strength to the elongate shaft 12, thereby allowing the percutaneous circulatory support device 10 to be advanced through tortuous anatomy.

An electrical cable 22 may extend from the junction housing 14 to a connector 24 at a proximal end thereof. The connector 24 may be configured to be connected to a controller (not shown) for controlling the blood pump 50, such as providing electrical power to the blood pump 50. The percutaneous circulatory support device 10 may also include an extension 26 connectable to the controller for sending and/or receiving signals, such as from one or more sensors during operation of the blood pump 50. Electrical wires for powering a motor to the blood pump and/or sensor wire for powering and/or sending/receiving sensor signals may extend through the elongate shaft 12 distal of the junction housing 14 and be connected to the electrical cable 22 and/or the extension 26.

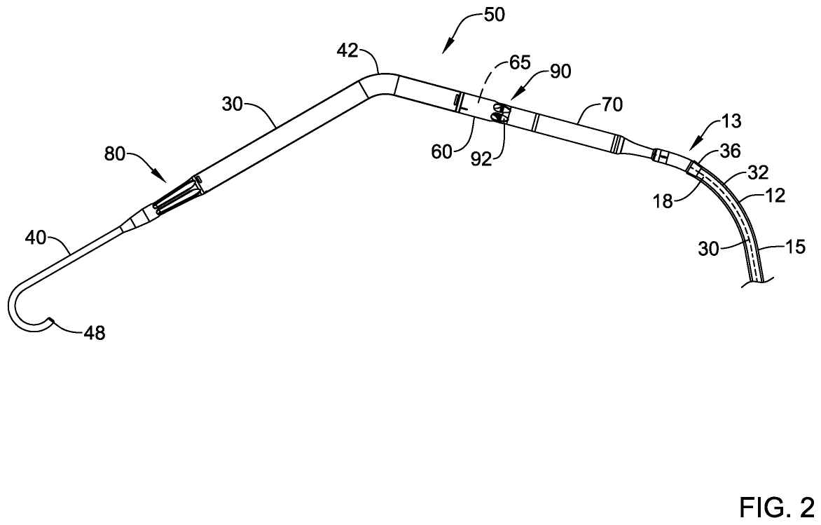

Additional features of the blood pump 50 are illustrated in FIG. 2. The blood pump 50 may generally include a flexible cannula 30, an impeller housing 60, and a motor housing 70. In some instances, the bend 42 may be a pre-formed bend formed in a medial region of the flexible cannula 30. In some embodiments, the flexible cannula 30, the impeller housing 60 and/or the motor housing 70 may be integrally or monolithically constructed. In other instances, the flexible cannula 30, the impeller housing 60 and/or the motor housing 70 may be separate components. The impeller housing 60 carries an impeller assembly 65 therein. The impeller assembly 65 may include an impeller secured to an impeller shaft that rotates relative to the impeller housing 60 to drive blood through the blood pump 50. In some embodiments, the impeller shaft, and the impeller of the impeller assembly 65 may be integrally formed, whereas, in other embodiments the impeller shaft and the impeller may be separate components.

Rotation of the impeller causes blood to flow from a blood inlet 80 of the blood pump 50, such as at a distal end of the flexible cannula 30, through the flexible cannula 30 and the impeller housing 60, and out of a blood outlet 90 proximal of the impeller, such as through a sidewall formed on the impeller housing 60. In some instances, the blood inlet 80 may include a plurality of blood inlet windows arranged around a circumference of the blood pump 50 (e.g., the flexible cannula 30). In some instances, the blood outlet 90 may include a plurality of blood outflow windows 92 arranged around a circumference of the impeller housing 60. In other embodiments, the inlet 80 and/or the outlet 90 may be formed on other portions of the blood pump 50.

With continued reference to FIG. 2, the motor housing 70 carries a motor configured to rotatably drive the impeller 65 of the impeller assembly relative to the impeller housing 60. Electrical power may be supplied to the motor through wiring extending through the elongate shaft 12, for example. In some instances, the motor may be physically connected to the impeller 65. For example, in some embodiments the impeller 65 may be mounted on the drive shaft of the motor. In other embodiments, the impeller shaft may be directly or indirectly coupled to the drive shaft of the motor. In some instances, the drive assembly may include a magnetic coupling between the motor and the impeller 65. For example, a driving magnet may be mounted on the drive shaft of the motor. Rotation of the driving magnet causes rotation of a driven magnet, which is connected to the impeller 65. More specifically, in embodiments incorporating an impeller shaft, the impeller shaft and the impeller 65 of the impeller assembly are configured to rotate with the driven magnet. In other embodiments, the motor may be coupled to the impeller assembly via other components.

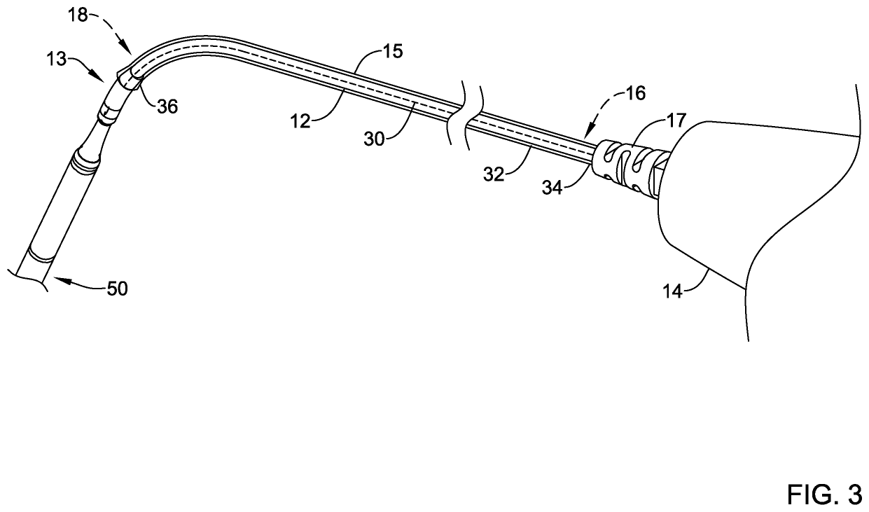

Additional features of the outer sheath 15 and the elongate shaft 12 are shown in FIG. 3. In at least a first configuration, the outer sheath 15 may generally surround, or be disposed around the elongate shaft 12. The outer sheath 15 may have an outer diameter greater than the outer diameter of the elongate shaft 12. For example, the outer sheath 15 may have an outer diameter of about 11 Fr, or 3.7 millimeters (mm) (0.144 inches), and the elongate shaft 12 may have an outer diameter of about 6 Fr, or 2 mm (0.079 inches); the outer sheath 15 may have an outer diameter of about 11 Fr, or 3.7 mm (0.144 inches), and the elongate shaft 12 may have an outer diameter of about 7 Fr, or 2.3 mm (0.092 inches); the outer sheath 15 may have an outer diameter of about 14 Fr, or 4.7 mm (0.184 inches), and the elongate shaft 12 may have an outer diameter of about 6 Fr, or 2 mm (0.079 inches); or the outer sheath 15 may have an outer diameter of about 14 Fr, or 4.7 mm (0.184 inches), and the elongate shaft 12 may have an outer diameter of about 7 Fr, or 2.3 mm (0.092 inches). In other examples, the outer sheath 15 may have an outer diameter between about 10 Fr, or 3.3 mm (0.131 inches) to about 14 Fr, or 4.7 mm (0.184 inches), and the elongate shaft 12 may have an outer diameter of about 5 Fr, or 1.7 mm (0.066 inches) to about 7 Fr, or 2.3 mm (0.092 inches). In some embodiments the elongate shaft 12 may have an outer diameter of about 7 Fr (2.3 mm or 0.092 inches) or less, or an outer diameter of 6 Fr (2 mm or 0.079 inches) or less, while the outer sheath 15 may have an outer diameter of 10 Fr (3.3 mm or 0.131 inches) or more, 11 Fr (3.7 mm or 0.144 inches) or more, 12 Fr (4 mm or 0.157 inches) or more, 13 Fr (4.3 mm 0.171 inches) or more, or 14 Fr (4.7 mm or 0.184 inches) or more. The walls of the outer sheath 15 may have a thickness between about 0.5 mm (0.020 inches) to about 1.2 mm (0.05 inches), between about 0.6 mm (0.024 inches) to about 1.15 mm (0.045 inches), between about 0.6 mm (0.024 inches) to about 1.1 mm (0.043 inches), between about 0.75 mm (0.030 inches) to about 1.2 mm (0.05 inches), or about 0.75 mm (0.030 inches) to about 1.1 mm (0.043 inches), in some instances. In some embodiments, the outer sheath 15 has an inner diameter larger than the outer diameter of the elongate member 12, such that there is a gap, or space 32 between the elongate member 12 and the outer sheath 15. In some embodiments, the gap, or space 32 between the outer sheath 15 and the elongate shaft 12 may be between about 0.035 mm (0.001 inches) to about 0.22 mm (0.009 inches), or about 0.1 mm (0.004 inches) to about 0.2 mm (0.008 inches). For instance, the outer sheath 15 may have an inner diameter of about 7 Fr or less (i.e., about 2.3 mm (0.092 inches) or less), or 6.5 Fr or less (i.e., about 2.17 mm (0.085 inches) or less). Other dimensions of the elongate shaft 12 and outer sheath 15 are contemplated.

The outer diameter of the outer sheath 15 may be less than the outer diameter of the blood pump 50, for example, less than the outer diameter of the motor housing 70, the impeller housing 60 and/or the cannula 30. Accordingly, the outer sheath 15 may be configured to be contained between the blood pump 50 and the junction box 14.

In some embodiments, there may be a fluid management system configured to flush or supply fluid to the space 32 between the outer sheath 15 and elongate shaft 12. In some embodiments, the outer sheath 15 may be tightly disposed around the elongate shaft 12 such that an inner surface of the outer sheath 15 contacts an outer surface of the elongate member 12. Alternatively, or additionally to other embodiments discussed herein, the distal end 36 of the outer sheath 15 may be releasably coupled to a proximal portion 13 of the blood pump 50. In some embodiments, and as illustrated, the distal end 36 of the outer sheath 15 may have a flared, tapered, or funneled shape, shaped and sized to fit around an enlarged proximal end or ferrule 13 of the blood pump 50. When intact, the outer sheath 15 may extend substantially the entire length of the elongate shaft 12 from the proximal portion or ferrule 13 of the blood pump to the connector or strain relief 17 on the distal side of the junction box 14. In some instances, the elongate shaft 12 may have a length of 60 centimeters (cm) (23.6 inches) or more, 70 cm (27.6 inches) or more, or 80 cm (31.5 inches) or more, and the outer sheath 15 may have a length of 95% or more, 98% or more, or 100% or more of the length of the elongate shaft 12. For instance, in some instances, the outer sheath 15 may have a length of 60 cm (23.6 inches) or more, 70 cm (27.6 inches) or more, or 80 cm (31.5 inches) or more. The outer sheath 15 may be positioned around the elongate shaft 12 such that the outer sheath 15 is not axially moveable relative to the elongate shaft 12. The outer sheath 15 may be positioned around the elongate shaft 12 such that the outer sheath 15 is not removable therefrom unless split along its length to be removed laterally from the elongate shaft, as will be described further herein.

The outer sheath 15 may be disposed between and abut the blood pump 50 and junction box 14 (e.g., the connector or strain relief 17 of the junction box 14). These components of the percutaneous circulatory support device 10 prevent proximal and distal movement of the elongate sheath 15 when it is intact. Said differently, the diameter (e.g., outer diameter and/or inner diameter) of the outer sheath 15 is smaller than the blood pump 50 and the junction box 14, precluding removal of the outer sheath 15 without slitting the outer sheath 15 as described further herein. In some instances, the outer sheath 15 may surround the elongate shaft 12, but not be fixedly secured to the elongate shaft 12. In some instances, the outer sheath 15 may be unsecured to the elongate shaft 12 along the entire length, or substantially the entire length of the outer sheath 15. In some embodiments, the outer sheath 15 may be releasably attached to the elongate shaft 12.

When advancing the blood pump 50 through the vasculature of a patient to position the blood pump 50 in the heart, the outer sheath 15 may be advanced distally with distal advancement of the elongate shaft 12. Said differently, the outer sheath 15 and the elongate shaft 12 may be distally advanced together within the vasculature to provide sufficient column strength to prevent buckling of the elongate shaft 12. The elongate shaft 12 and the outer sheath 15 may be advanced until the blood pump 50 is positioned across the aortic valve, at which point the distal end 36 of the outer sheath 15 (located proximate the proximal end of the blood pump 50) may be positioned in the aorta of the patient, for example, while the proximal end 34 of the outer sheath 15 is located exterior of the patient.

The outer sheath 15 includes a longitudinal separation line 30 (or a plurality of longitudinal separation lines 30), or an area of increased weakness compared to the rest of the sheath 15, providing a preferential region of separation of the outer sheath therealong. The longitudinal separation line 30 may be formed by any method known in the art, for example, extrusion, scoring, molding, perforating, laser cutting, heating, cutting or the like. The longitudinal separation line 30 may allow for removal of the outer sheath 15 after the percutaneous circulatory support device 10 has been placed within a patient's vasculature, leaving the elongate shaft 12 extending through the vasculature of the patient from the blood pump 50, out through a vascular access site, and to the junction box 14 located exterior of the patient.

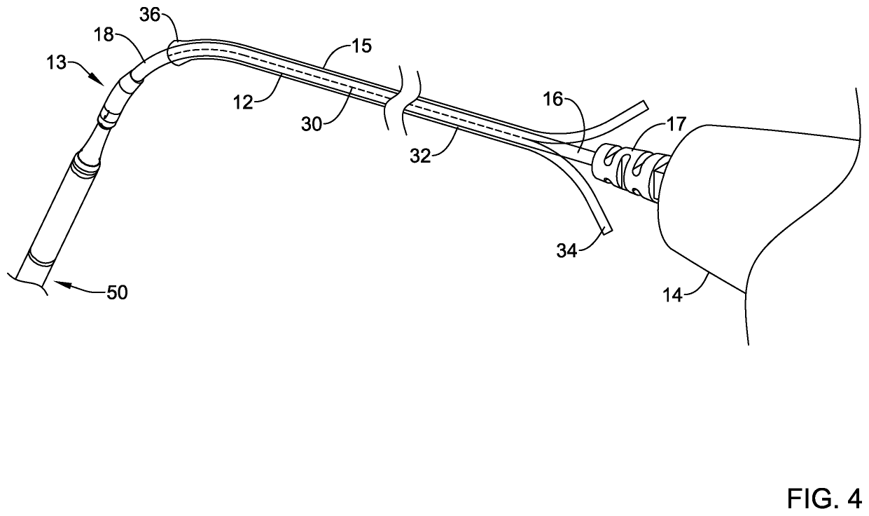

FIG. 4, shows a second configuration of the outer sheath 15 during removal from the elongate shaft 12. After the blood pump 50 has been positioned within a vasculature, it is advantageous to have a smaller diameter elongate shaft 12 taking up a smaller cross-sectional area of the vascular lumen that the elongate shaft 12 is disposed in, allowing for blood to flow freely around the elongate shaft 12. The larger diameter outer sheath 15 can be fully removed from the percutaneous circulatory support device 10, leaving the smaller diameter elongate shaft 12 in place. The outer sheath 15 is configured to split apart at the longitudinal separation line 30, facilitating smooth and efficient withdrawal of the sheath 15 from the vasculature. There may be any number of longitudinal separation lines 30 along the length of the sheath 15 configured to split the outer sheath 15 into separate pieces as it is removed. For example, and as illustrated in FIG. 4, there may be two longitudinal separation lines 30 along the length of the outer sheath 15, positioned on opposite sides of the outer sheath 15, such that the outer sheath 15 splits into two segments or semi-circular longitudinal strips as the longitudinal separation lines 30 are opened.

Removal of the outer sheath 15 does not affect the position of the percutaneous circulatory support device 10 or require any repositioning of the blood pump 50 within the patient. The separation of the outer sheath 15 into segments allows for the percutaneous circulatory support device 10, and components thereof to pass laterally through the separated portions of the outer sheath 15 as the outer sheath 15 is removed. The outer sheath 15 can be made of a low friction material to ease removal. Additionally, the space 32 between the outer sheath 15 and the elongate shaft 12 may ease in removal. To initiate removal of the outer sheath 15, a user may pull the sheath 15 proximally, leaving the elongate shaft 12 in place as to not shift the position of the blood pump 50 within the heart (e.g., positioned across the aortic valve). Since the proximal end 34 of the outer sheath 15 abuts the connector or strain relief 17 of the junction box 14, the outer sheath 15 cannot move unless the longitudinal separation line 30 begins to split open. Thus, the outer sheath 15 will begin to split at the longitudinal separation line 30 when withdrawn proximally. In some embodiments, the longitudinal separation line 30 may be opened as the proximal end 34 of the sheath 15 interacts with the connector or strain relief 17. Once the longitudinal separation line 30 of the outer sheath 15 is opened at the proximal end 34, the outer sheath 15 can move relative to the elongate shaft 12. Continued proximal withdrawal of the outer sheath 15 propagates the separation of the outer sheath 15 distally along the longitudinal separation line(s) 30 toward the distal end 36 of the outer sheath 15. Propagation of the separation of the outer sheath 15 along the longitudinal separation line(s) 30 may continue to the distal end 36 of the outer sheath 15, at which point the outer sheath 15 has been separated into multiple (e.g., two) pieces. Other methods of withdrawal are contemplated, for example, the outer sheath 15 may include tabs or handles allowing a user to grip and pull the sheath apart to initiate removal. Such tabs or handles may be provided proximate the proximal end 34 of the outer sheath 15 to facilitate exerting a lateral force on the proximal end 34 of the outer sheath 15 to initiate lateral separation of the outer sheath 15 along the longitudinal separation line(s) 30.

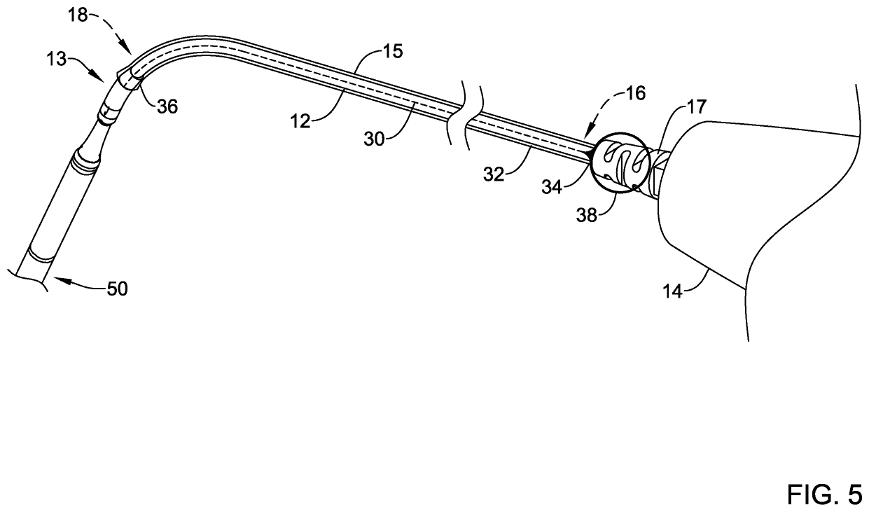

In other embodiments, and as illustrated in FIG. 5, the outer sheath 15, includes a grip 38 (e.g., tab, handle, loop, lever, etc.) for initiating removal of the outer sheath 15 from the elongate shaft 12. The grip 38 may be coupled to a ripcord, or a portion of the outer sheath 15, such that pulling on the grip 38 opens the outer sheath 15 at one or more longitudinal separation lines 30 to split the sheath 15 into segments. In some embodiments, there may be multiple grips 38, to allow a user to apply opposing lateral forces to the proximal end 34 of the sheath 15. In some embodiments, the grip(s) 38 or other features such as grooves, indents etc. may assist a user with gripping and pulling the outer sheath 15 proximally during removal.

It will be understood that the dimensions described in association with the above figure are illustrative only, and that other dimensions of slits and filter sheaths are contemplated. The materials that can be used for the various components of the percutaneous circulatory support device (and/or other systems or components disclosed herein) and the various elements thereof disclosed herein may include those commonly associated with medical devices. For simplicity purposes, the following discussion makes reference to the percutaneous circulatory support device (and variations, systems or components disclosed herein). However, this is not intended to limit the devices and methods described herein, as the discussion may be applied to other elements, members, components, or devices disclosed herein.

In some embodiments, the percutaneous circulatory support device (and variations, systems or components thereof disclosed herein) may be made from a metal, metal alloy, ceramics, zirconia, polymer (some examples of which are disclosed below), a metal-polymer composite, combinations thereof, and the like, or other suitable material. Some examples of suitable metals and metal alloys include stainless steel, such as 444V, 444L, and 314LV stainless steel; mild steel; nickel-titanium alloy such as linear-elastic and/or super-elastic nitinol; cobalt chromium alloys, titanium and its alloys, alumina, metals with diamond-like coatings (DLC) or titanium nitride coatings, other nickel alloys such as nickel-chromium-molybdenum alloys (e.g., UNS: N06625 such as INCONEL® 625, UNS: N06022 such as HASTELLOY® C-22®, UNS: N10276 such as HASTELLOY® C276®, other HASTELLOY® alloys, and the like), nickel-copper alloys (e.g., UNS: N04400 such as MONEL® 400, NICKELVAC® 400, NICORROS® 400, and the like), nickel-cobalt-chromium-molybdenum alloys (e.g., UNS: R44035 such as MP35-N® and the like), nickel-molybdenum alloys (e.g., UNS: N10665 such as HASTELLOY® ALLOY B2®), other nickel-chromium alloys, other nickel-molybdenum alloys, other nickel-cobalt alloys, other nickel-iron alloys, other nickel-copper alloys, other nickel-tungsten or tungsten alloys, and the like; cobalt-chromium alloys; cobalt-chromium-molybdenum alloys (e.g., UNS: R44003 such as ELGILOY®, PHYNOX®, and the like); platinum enriched stainless steel; titanium; platinum; palladium; gold; combinations thereof; and the like; or any other suitable material.

As alluded to herein, within the family of commercially available nickel-titanium or nitinol alloys, is a category designated “linear elastic” or “non-super-elastic” which, although may be similar in chemistry to conventional shape memory and super elastic varieties, may exhibit distinct and useful mechanical properties. Linear elastic and/or non-super-elastic nitinol may be distinguished from super elastic nitinol in that the linear elastic and/or non-super-elastic nitinol does not display a substantial “super-elastic plateau” or “flag region” in its stress/strain curve like super elastic nitinol does. Instead, in the linear elastic and/or non-super-elastic nitinol, as recoverable strain increases, the stress continues to increase in a substantially linear, or a somewhat, but not necessarily entirely linear relationship until plastic deformation begins or at least in a relationship that is more linear than the super elastic plateau and/or flag region that may be seen with super elastic nitinol. Thus, for the purposes of this disclosure linear elastic and/or non-super-elastic nitinol may also be termed “substantially” linear elastic and/or non-super-elastic nitinol.

In some cases, linear elastic and/or non-super-elastic nitinol may also be distinguishable from super elastic nitinol in that linear elastic and/or non-super-elastic nitinol may accept up to about 2-5% strain while remaining substantially elastic (e.g., before plastically deforming) whereas super elastic nitinol may accept up to about 8% strain before plastically deforming. Both of these materials can be distinguished from other linear elastic materials such as stainless steel (that can also be distinguished based on its composition), which may accept only about 0.2 to 0.44 percent strain before plastically deforming.

In some embodiments, the linear elastic and/or non-super-elastic nickel-titanium alloy is an alloy that does not show any martensite/austenite phase changes that are detectable by differential scanning calorimetry (DSC) and dynamic metal thermal analysis (DMTA) analysis over a large temperature range. For example, in some embodiments, there may be no martensite/austenite phase changes detectable by DSC and DMTA analysis in the range of about −60 degrees Celsius (°C) to about 120 °C in the linear elastic and/or non-super-elastic nickel-titanium alloy. The mechanical bending properties of such material may therefore be generally inert to the effect of temperature over this very broad range of temperature. In some embodiments, the mechanical bending properties of the linear elastic and/or non-super-elastic nickel-titanium alloy at ambient or room temperature are substantially the same as the mechanical properties at body temperature, for example, in that they do not display a super-elastic plateau and/or flag region. For example, across a broad temperature range, the linear elastic and/or non-super-elastic nickel-titanium alloy maintains its linear elastic and/or non-super-elastic characteristics and/or properties.

In some embodiments, the linear elastic and/or non-super-elastic nickel-titanium alloy may be in the range of about 50 to about 60 weight percent nickel, with the remainder being essentially titanium. In some embodiments, the composition is in the range of about 54 to about 57 weight percent nickel. One example of a suitable nickel-titanium alloy is FHP-NT alloy commercially available from Furukawa Techno Material Co. of Kanagawa, Japan. Other suitable materials may include ULTANIUM™ (available from Neo-Metrics) and GUM METAL™ (available from Toyota). In some other embodiments, a super-elastic alloy, for example a super-elastic nitinol can be used to achieve desired properties.

In at least some embodiments, portions or all of the percutaneous circulatory support device (and variations, systems or components thereof disclosed herein) may also be doped with, made of, or otherwise include a radiopaque material. Radiopaque materials are understood to be materials capable of producing a relatively bright image on a fluoroscopy screen or another imaging technique during a medical procedure. This relatively bright image aids a user in determining the location of the percutaneous circulatory support device (and variations, systems or components thereof disclosed herein). Some examples of radiopaque materials can include, but are not limited to, gold, platinum, palladium, tantalum, tungsten alloy, polymer material loaded with a radiopaque filler, and the like. Additionally, other radiopaque marker bands and/or coils may also be incorporated into the design of the percutaneous circulatory support device (and variations, systems or components thereof disclosed herein) to achieve the same result.

In some embodiments, the percutaneous circulatory support device (and variations, systems or components thereof disclosed herein) and/or portions thereof, may be made from or include a polymer or other suitable material. Some examples of suitable polymers may include polytetrafluoroethylene (PTFE), ethylene tetrafluoroethylene (ETFE), fluorinated ethylene propylene (FEP), polyoxymethylene (POM, for example, DELRIN® available from DuPont), polyether block ester, polyurethane (for example, Polyurethane 85A), polypropylene (PP), polyvinylchloride (PVC), polyether-ester (for example, ARNITEL® available from DSM Engineering Plastics), ether or ester based copolymers (for example, butylene/poly(alkylene ether) phthalate and/or other polyester elastomers such as HYTREL® available from DuPont), polyamide (for example, DURETHAN® available from Bayer or CRISTAMID® available from Elf Atochem), elastomeric polyamides, block polyamide/ethers, polyether block amide (PEBA, for example available under the trade name PEBAX®), ethylene vinyl acetate copolymers (EVA), silicones, polyethylene (PE), MARLEX® high-density polyethylene, MARLEX® low-density polyethylene, linear low density polyethylene (for example REXELL®), polyester, polybutylene terephthalate (PBT), polyethylene terephthalate (PET), polytrimethylene terephthalate, polyethylene naphthalate (PEN), polyetheretherketone (PEEK), polyimide (PI), polyetherimide (PEI), polyphenylene sulfide (PPS), polyphenylene oxide (PPO), poly paraphenylene terephthalamide (for example, KEVLAR®), polysulfone, nylon, nylon-12 (such as GRILAMID® available from EMS American Grilon), perfluoro(propyl vinyl ether) (PFA), ethylene vinyl alcohol, polyolefin, polystyrene, epoxy, polyvinylidene chloride (PVdC), poly(styrene-b-isobutylene-b-styrene) (for example, SIBS and/or SIBS 50A), polycarbonates, ionomers, polyurethane silicone copolymers (for example, Elast-Eon® from AorTech Biomaterials or ChronoSil® from AdvanSource Biomaterials), biocompatible polymers, other suitable materials, or mixtures, combinations, copolymers thereof, polymer/metal composites, and the like. In some embodiments, the sheath can be blended with a liquid crystal polymer (LCP). For example, the mixture can contain up to about 6 percent LCP.

In some embodiments, the percutaneous circulatory support device (and variations, systems or components thereof disclosed herein) may include and/or be treated with a suitable therapeutic agent. Some examples of suitable therapeutic agents may include anti-thrombogenic agents (such as heparin, heparin derivatives, urokinase, and PPack (dextrophenylalanine proline arginine chloromethyl ketone)); anti-proliferative agents (such as enoxaparin, angiopeptin, monoclonal antibodies capable of blocking smooth muscle cell proliferation, hirudin, and acetylsalicylic acid); anti-inflammatory agents (such as dexamethasone, prednisolone, corticosterone, budesonide, estrogen, sulfasalazine, and mesalamine); antineoplastic/antiproliferative/anti-mitotic agents (such as paclitaxel, 5-fluorouracil, cisplatin, vinblastine, vincristine, epothilones, endostatin, angiostatin and thymidine kinase inhibitors); anesthetic agents (such as lidocaine, bupivacaine, and ropivacaine); anti-coagulants (such as D-Phe-Pro-Arg chloromethyl ketone, an RGD peptide-containing compound, heparin, anti-thrombin compounds, platelet receptor antagonists, anti-thrombin antibodies, anti-platelet receptor antibodies, aspirin, prostaglandin inhibitors, platelet inhibitors, and tick antiplatelet peptides); vascular cell growth promoters (such as growth factor inhibitors, growth factor receptor antagonists, transcriptional activators, and translational promoters); vascular cell growth inhibitors (such as growth factor inhibitors, growth factor receptor antagonists, transcriptional repressors, translational repressors, replication inhibitors, inhibitory antibodies, antibodies directed against growth factors, bifunctional molecules consisting of a growth factor and a cytotoxin, bifunctional molecules consisting of an antibody and a cytotoxin); cholesterol-lowering agents; vasodilating agents; and agents which interfere with endogenous vascoactive mechanisms.

It should be understood that this disclosure is, in many respects, only illustrative. Changes may be made in details, particularly in matters of shape, size, and arrangement of steps without exceeding the scope of the disclosure. This may include, to the extent that it is appropriate, the use of any of the features of one example embodiment being used in other embodiments. The disclosure's scope is, of course, defined in the language in which the appended claims are expressed.

What is claimed is:

1. A percutaneous circulatory support device comprising:
a blood pump;
an elongate shaft extending proximally from the blood pump; and
an outer sheath having a proximal end, a distal end, and one or more longitudinal separation lines extending along a length of the outer sheath;
wherein in a first configuration, the elongate shaft is disposed within a lumen of the outer sheath; and
wherein in a second configuration, the outer sheath is configured to be split along the one or more longitudinal separation lines and withdrawn from over the elongate shaft.

2. The percutaneous circulatory support device of claim 1, wherein the outer sheath is configured to be split into two pieces.

3. The percutaneous circulatory support device of claim 1, wherein the one or more longitudinal separation lines are one or more score lines of reduced wall thickness.

4. The percutaneous circulatory support device of claim 1, wherein when in the first configuration, the outer sheath is not axially moveable relative to the elongate shaft.

5. The percutaneous circulatory support device of claim 1, wherein the distal end of the outer sheath has a flared shape configured to fit around a proximal end of the blood pump.

6. The percutaneous circulatory support device of claim 1, further comprising a grip disposed on the proximal end of the outer sheath, wherein proximal withdrawal of the grip initiates proximal retraction and dividing of the outer sheath into multiple pieces.

7. The percutaneous circulatory support device of claim 6, wherein the grip is connected to a ripcord configured to open the one or more longitudinal separation lines of the outer sheath.

8. The percutaneous circulatory support device of claim 1, wherein an inner diameter of the outer sheath is greater than the outer diameter of the elongate shaft.

9. The percutaneous circulatory support device of claim 1, wherein the one or more longitudinal separation lines define an area of weakness providing a preferential separation region.

10. The percutaneous circulatory support device of any one of claim 1, wherein the outer sheath has a length of 60 cm or more.

11. A percutaneous circulatory support device comprising:
a blood pump;
an elongate shaft extending proximally from the blood pump; and
an outer sheath surrounding the elongate shaft, the outer sheath having a proximal end proximate a proximal end of the elongate shaft, a distal end proximate a distal end of the elongate shaft, and one or more longitudinal separation lines extending along a length of the outer sheath;
wherein the outer sheath is configured to be split along the one or more longitudinal separation lines and withdrawn from over the elongate shaft.

12. The percutaneous circulatory support system of claim 11, wherein the outer sheath is not fixedly secured relative to the elongate shaft.

13. The percutaneous circulatory support system of claim 11, wherein the outer sheath has a length of 60 cm or more.

14. The percutaneous circulatory support system of claim 11, wherein the distal end of the outer sheath is proximate a proximal end of the blood pump.

15. The percutaneous circulatory support system of claim 11, wherein an inner diameter of the outer sheath is greater than an outer diameter of the elongate shaft.

16. A method of placing a percutaneous circulatory support device within a vasculature, the method comprising:
advancing a percutaneous circulatory support device through a vasculature of a patient, the percutaneous circulatory support device including:
a blood pump;
an elongate shaft extending proximally from the blood pump; and
an outer sheath surrounding the elongate shaft, the outer sheath having a proximal end proximate a proximal end of the elongate shaft, a distal end proximate a distal end of the elongate shaft, and one or more longitudinal separation lines extending along a length of the outer sheath;
positioning the blood pump at a desired treatment location;
thereafter, removing the outer sheath from the elongate shaft with the blood pump at the desired treatment location.

17. The method of claim 16, wherein the outer sheath further comprises at least one longitudinal separation line extending along a length of the outer sheath.

18. The method of claim 17, further comprising dividing the outer sheath along the at least one longitudinal separation line to remove the outer sheath from the elongate shaft.

19. The method of claim 18, wherein the outer sheath is not fixedly secured relative to the elongate shaft.

20. The method of claim 17, wherein the blood pump is maintained at the desired treatment location after removal of the outer sheath from the elongate shaft.
